(19) United States
(12) Patent Application Publication
(10) Pub. No.: US 2015/0174222 A1
(43) Pub. Date: Jun. 25, 2015

(54) MODIFICATION OF THE IMMUNOMODULATORY EFFECTS OF CELLS

(71) Applicant: Centre National De La Recherche Scientifique, Paris (FR)

(72) Inventors: Louis Casteilla, Escalquens (FR); Roxane Blattes, Toulouse (FR); Yannick Jeanson, Revel (FR); Karin Tarte, Rennes (FR)

(73) Assignee: Centre National De La Recherche Scientifique, Paris (FR)

(21) Appl. No.: 14/409,049

(22) PCT Filed: Jun. 21, 2013
(86) PCT No.: PCT/IB2013/055111

§ 371 Date: Dec. 18, 2014

(30) Foreign Application Priority Data
Jun. 22, 2012 (FR) 1255957

(51) Int. Cl. A61K 39/00; C07K 16/18; C07K 14/47; C12N 15/113; C12N 5/077

(52) U.S. Cl. CPC A61K 39/0005; C12N 15/113; C12N 5/0653; C07K 14/47; A61K 39/0011; C07K 16/18; C12N 2310/14; C12N 2503/02

(57) ABSTRACT

The present invention relates to an isolated cell having immunomodulatory potential, in which the expression and/or activity of periostin is modulated, or to the culture supernatant of said cell, for the use thereof as an immunosuppressive or immunostimulatory drug. The present invention also relates to periostin for the use thereof as an immunostimulatory drug or to a periostin inhibitor for the use thereof as an immunosuppressive drug.

The present invention relates to the fields of cell immunotherapy, and more particularly the use of mammalian cells having immunomodulatory potential and which have been genetically or pharmacologically modified, as an immunosuppressive or immunostimulatory medicament. The present invention also relates to novel molecules having an immunosuppressive or immunostimulatory effect.

Cell therapy consists of the injection in a subject of autologous or allogenic live cells, with the aim of treating or preventing a disease or of reconstructing a damaged tissue. These cells can be stem cells, progenitor or precursor cells, or functional differentiated cells from the blood or from a tissue. These cells can also be genetically transformed so as to express a transgene of therapeutic interest, in a tissue. In addition, the genetic modification of these cells can improve their survival, their metabolic characteristics, their proliferative capacities or, for stem cells or precursors, their differentiation capacities. These same objectives can be obtained by preconditioning these cells using pharmacological tools.

Among stem cells, a distinction is made between:
embryonic stem cells (ESCs) which come from an embryo at an early stage (from the zygote to the blastomere). These cells are totipotent, i.e. they are capable of differentiating into any tissue of the organism, and are capable of self-renewing;
adult stem cells which are present in most tissues of the organism. These cells are either pluripotent, i.e. they are capable of forming all the cell types except the embryonic appendages, such as induced pluripotent stem cells or “Multilineage-differentiating Stress Enduring Cells”; or multipotent, i.e. they are capable of forming various types of cells of a given cell lineage; or unipotent, i.e. they can form just one cell type.

Progenitor and precursor cells are derived from stem cells, i.e. they are more engaged in a differentiation pathway than the stem cells. They are also capable of forming one or more cell types, but are not capable of self-renewing.

Mesenchymal stem cells (MSCs)—also called mesenchymal stromal cells—are currently the subject of numerous research studies for their therapeutic applications. In the description which follows, the terms “mesenchymal stem cell” and “mesenchymal stromal cell” are used without distinction. These adult stem cells were initially isolated and characterized from bone marrow mononuclear cells (BM-MSCs), but can also be isolated from adipose tissue, from the skin, from the spleen and from the heart. MSCs have phenotypic characteristics, for example CD45⁻, CD34^{+/-} (depending on the tissue origin and their proliferation stage), CD13⁺, which makes it possible to distinguish them from hematopoietic stem cells which are CD45⁺, CD34⁺, CD13⁻. Depending on the inducers used, they have an osteogenic, adipogenic, chondrogenic, myogenic and angiogenic differentiation potential for example. They also have a paracrine activity and are capable of secreting growth factors, pro-inflammatory or anti-inflammatory cytokines, chemokines and prostaglandins (for review, see Le Blanc 2006, Kode et al., 2009, Hoogduijn et al., 2010 and Dazzi et al., 2011). Consequently, they also exhibit great similarities with monocytes and macrophages (Charrie\grave{r}e et al., 2006) and also fibroblasts (Hannifa et al., 2007) which have similar immunomodulatory properties. MSCs are thus used in regenerative cell therapy for their properties of multiple differentiation and also for their proliferative, angiogenic, anti-apoptotic, trophic and immunomodulatory properties. For example, Le Blanc et al. (2008) have shown in humans that mesenchymal stem cells combined with a graft of hematopoietic stem cells can reduce the risk of acute graft versus host disease (GVHD) in allografts.

MSCs isolated from adipose tissue are called ASCs, ADSCs (adipose derived stem/stroma cells), ADASCs (adipose tissue-derived adult stem cells) or AD-MSCs (adipose-derived MSCs). Adipose tissue has the advantage of being easily obtained by liposuction under local anesthetic and of containing several populations of immature cells, including a high majority of ASCs. The ASCs are then isolated and purified after proteolytic digestion of the white adipose tissue (e.g., with collagenase) and selection by means of a step of adhesion on a plastic substrate (for review, see Gimble et al., 2007), or can be directly selected on the basis of their surface phenotype (for example, selection of CD45⁻, CD34⁺ and CD31⁻ cells). Although they have specific characteristics, ASCs show many characteristics that are common with mesenchymal stem cells derived from the bone marrow, including the paracrine activity and the immunomodulatory properties (Planat-Bénard et al., 2004, Puissant et al., 2005, Yañez et al., 2006, González et al., 2009a and 2009b, Constantin et al., 2009 and Yoo et al., 2009). In addition, insofar as their self-renewal has not been clearly established, the term “mesenchymal stromal cells” should be used to describe them (Casteilla et al., 2011). Nevertheless, these cells can serve as a cell model for all mesenchymal stem cells. ASCs are currently studied at the clinical level in several types of applications (for review, see Casteilla et al., 2011), including critical ischemia of the lower limbs and the treatment of fistulae which may or may not be associated with Crohn's disease (Garcia-Olmo et al., 2009).

Despite encouraging preclinical and clinical results regarding the use of MSCs in the context of cell therapies (for review, see Uccelli et al., 2008), the immunomodulatory potential of MSCs is sometimes too weak to obtain good results in the treatment of diseases or dysfunctions involving inflammation, such as chronic inflammatory diseases or autoimmune diseases. There is therefore a need to improve the immunomodulatory potential of MSCs, thus making it possible to reduce the number of cells required for treatment and/or to improve their effectiveness, thereby reducing accordingly the amount of cells initially sampled and necessary for obtaining grafted MSCs, and also the time for culturing the cells.

Periostin (POSTN or PN) is an extracellular matrix adhesion protein, secreted in particular by osteoblasts and preferentially expressed in the periosteum of bones and the periodontal ligaments of teeth (for review, see Kudo, 2011 and Frangogianni, 2012). Periostin is also expressed in other tissues, such as the heart, the mammary glands, bone marrow-derived mesenchymal stromal cells (Coutu et al., 2008) and certain cancer cells. The nucleotide and peptide sequences of the four known isoforms of periostin are available in the GENBANK database, under the accession numbers GI:209863034 (NP_{—}001129408.1; isoform 1), GI:209862911 (NP_{—}001129406.1; isoform 2), GI:209863011 (NP_{—}001129407.1; isoform 3) and GI:209863034 (NP_{—}001129408.1; isoform 4). Periostin contains, from its N-terminal end to its C-terminal end: a secretion signal sequence, a cysteine-rich domain (EMI domain), 4 homologous repeat regions (Fasciclin I (FAS1) domains) and a hydrophobic domain. The FAS1 domains of proteins are well known to those skilled in the art; they are referenced, for example, in the EMBL-EBI database under the accession number IPR000782 or in the PFAM database under the accession number PF02469. The FAS1 domains of periostin have in particular been described by Coutu et al., 2008. Periostin maintains the structure and the integrity of the support tissues (collagen) and participates in bone growth. Kudo et al. (2004) have shown, in the zebra fish, that the inhibition of periostin mRNA translation with an antisense morpholino oligonucleotide inhibits myoseptum formation in the embryo. Rios et al. (2005) have shown, using “knock-in” transgenic mice which do not express periostin, that periostin is required for maintaining the integrity of the periodontal ligament in response to a mechanical stress. Takayama et al. (2006) have shown that the expression of periostin is induced by the cytokines TGF-β and/or IL-4 and IL-13 expressed in response to inflammation or to a mechanical stress. In addition, increased periostin expression in tissue repair or remodeling and fibrosis processes may be due to local activation of TGF-β and of the bone morphogenetic protein (BMP) signaling pathway (for review, see Frangogianni, 2012). Moreover, it appears that periostin stimulates cell growth in several types of cancer, such as breast cancer. Orecchia et al. (2011) have shown in vitro that the treatment of SKMEL-28 cells with a blocking monoclonal antibody, directed against the YN motif located in the second FAS1 domain of human periostin, inhibits tumor growth and reduces tumor vascular density. Finally, international application WO 2010/025555 indicates that periostin can be used as a medicament for pancreatic tissue regeneration.

To the knowledge of the inventors, no piece of data links periostin to an immunomodulatory effect of cells expressing this protein.

The inventors have given themselves the aim of modifying the immunomodulatory potential of cells having an immunomodulatory potential, and more particularly mesenchymal stem/stromal cells.

The inventors have therefore shown that the inhibition of periostin expression in human adipose tissue-derived mesenchymal stromal cells (ASCs), which have not been genetically transformed, makes it possible to increase the immunosuppressive potential of these cells.

The inventors have also shown that the immunosuppressive potential of human adipose tissue-derived mesenchymal stromal cells (ASCs), which have not been genetically transformed, is inhibited when periostin is added to these cells. Increasing periostin expression in ASCs therefore makes it possible to reduce or inhibit the immunosuppressive potential of these cells, i.e. to confer an immunostimulatory potential on these cells.

From the viewpoint of these results obtained with ASCs used as model cells, the inventors have therefore demonstrated, unexpectedly, the role of periostin in the control of the paracrine activity of cells having an immunomodulatory potential, in particular of mesenchymal stem/stromal cells. These results also show that periostin can be used as an immunostimulatory medicament and that a periostin inhibitor can be used as an immunosuppressive medicament.

A subject of the present invention is an isolated diploid cell having an immunomodulatory potential, in which the expression and/or the activity of periostin is modulated, or the culture supernatant of said cell, for the use thereof as a medicament.

Preferably, said medicament is an immunosuppressive medicament or an immunostimulatory medicament.

The expression “cell having an immunomodulatory potential” is intended to mean a cell which makes it possible to decrease or increase the natural capacities of the immune system in an organism, i.e. to decrease (immunosuppression) the natural immune defenses when they may be harmful to said organism, or, on the contrary, to reinforce them (immunostimulation) when they are insufficient or depressed. This cell is characterized by its ability to act on the effectors of immunity. The immunomodulatory potential of a cell can be determined by those skilled in the art using well known techniques, such as immunophenotyping and more particularly and by way of example, for lymphocytes, the mixed lymphocyte reaction (MLR), but also the response under stimulation of neutrophils; for example, for mesenchymal stromal cells, see Perico et al., 2011; for dendritic cells, see Zhao et al., 2012; for NK cells, see Abdelrazik et al., 2011; for lymphocytes, see Perico et al., 2011, Najar et al., 2010, Zhou et al., 2011 and Kronsteiner et al., 2011. Said cell having an immunomodulatory potential (before modulation of the expression and/or the activity of periostin) expresses periostin. Preferably, said cell having an immunomodulatory potential also expresses at least one immunomodulatory molecule, such as IFN-β, IDO-1, TSG-6, HLA-G, PGE2, TGF-β, galectin, HO-1, IL-6, IL-1RA, IL-33, AIRE (“autoimmune regulator”), hEGF, TNF, GM-CSF and/or JAG1, which are well known to those skilled in the art, preferably IFN-β, IDO-1, TSG-6, HLA-G and IL-1RA. The measurement of the expression of these genes in a cell can be carried out by RT-PCR, as described in the examples hereinafter.

Advantageously, said cell having an immunomodulatory potential is chosen from a mesenchymal stromal cell, a progenitor cell, a precursor cell, a cell differentiated from a mesenchymal stromal cell, a macrophage, a monocyte, a mast cell, a myeloid cell, a fibroblast, a dendritic cell, a lymphocyte (for example a Treg lymphocyte), an NK cell, a lymphoid cell and a myoblast.

Further advantageously, said mesenchymal stromal cell is chosen from a mesenchymal stromal cell derived from bone marrow (BM-MSC), from adipose tissue (ASC or AD-MSC), from a solid tissue, from the placenta, from adult blood or from cord blood.

Preferably, said cell having an immunomodulatory potential is a mammalian cell, more preferably a human cell.

Of course, said cell having an immunomodulatory potential is a living cell.

In addition, said cell having an immunomodulatory potential is not a cancer cell.

The expression “modulating the expression and/or the activity of periostin” is intended to mean the modification of the expression and/or the activity of periostin with respect to a cell having an immunomodulatory potential, either by total or partial inhibition of the expression and/or of the activity of said periostin, including by inhibition of its signaling pathways, or by increasing the expression and/or the activity of said periostin (overexpression of said periostin or stimulation of the signaling pathways of said periostin).

The choice by those skilled in the art of inhibiting or increasing the expression and/or the activity of said periostin as previously indicated is made according to the result that it is desired to obtain in terms of immunomodulation, namely respectively stimulating the immunosuppressive or immunostimulatory effect of said cell having an immunomodulatory potential. Thus, if those skilled in the art wish to stimulate the immunosuppressive effect of a cell having an immunomodulatory potential, then it is necessary to inhibit the expression and/or the activity of said periostin, including the inhibition of its signaling pathways, in said cell. If those skilled in the art wish to stimulate the immunostimulatory effect of a cell having an immunomodulatory potential, then it is necessary to increase the expression and/or the activity of said periostin, including the stimulation of its signaling pathways, in said cell.

The immunosuppressive potential (or the immunosuppressive properties) or the immunostimulatory potential (or the immunostimulatory properties) of a cell according to the present invention can be determined by measuring the expression of the mRNAs of the genes involved in the immunomodulation, such as the genes encoding the proteins IFN-β, IDO-1, TSG-6, HLA-G, PGE2, TGF-β, galectin, HO-1, IL-6, IL-1RA, IL-33, AIRE, hEGF, TNF, GM-CSF and/or JAG1, or by measuring the content of these proteins in this cell.

According to one preferred embodiment of the invention, said isolated cell having an immunomodulatory potential, in which the expression and/or the activity of periostin is totally or partially inhibited, or the culture supernatant of this cell, is of use as an immunosuppressive medicament for the regeneration (reconstruction) of a tissue, or organ transplantation (for limiting rejection), such as kidney transplantation, or in the treatment:
of graft versus host disease (GVHD),
of chronic inflammatory bowel diseases, such as Crohn's disease, celiac disease and irritable bowel syndrome;
of chronic inflammatory rheumatism, such as arthritis, rheumatoid arthritis, ankylosing spondylarthritis and psoriatic arthritis;
of chronic inflammatory diseases of the central nervous system, such as multiple sclerosis and amyotrophic lateral sclerosis;
of lupus;
of autoimmune thyroiditis;
of complex anal fistulae;
of asthmatic reactions of type IV delayed hypersensitivity type;
of allergies;
of inflammatory scars, such as hypertrophic scars;
of tissue necroses;
of autoimmune diseases, such as autoimmune encephalitis, autoimmune colitis and systemic lupus erythematosus;
of ulcers;
of diabetes;
of microbial infections, due for example to a bacterium, a protozoan parasite or a virus.

According to another preferred embodiment of the invention, said isolated cell, in which the expression and/or the activity of periostin, including its signaling pathways, is increased, or the culture supernatant of this cell, is of use as an immunostimulatory medicament intended for vaccination, i.e. a vaccine adjuvant, or intended for the treatment:
of a cancer or of an infection associated with an immunodeficiency;
of a selective or combined immunoglobulin deficiency;
of an isolated T lymphocyte deficiency;
of a purine nucleoside phosphorylase deficiency;
of a severe combined immunodeficiency caused by adenosine deaminase deficiency;
of common variable hypogammaglobulinemia.

Periostin is well known to those skilled in the art. The amino acid sequences of the four isoforms of human periostin are available in the GENBANK database under the accession numbers GI:209863034 (NP_{—}001129408.1; isoform 1), GI:209862911 (NP_{—}001129406.1; isoform 2), GI:209863011 (NP_{—}001129407.1; isoform 3) and GI:209863034 (NP_{—}001129408.1; isoform 4).

For the purposes of the present invention, the term “periostin” is intended to mean these four isoforms and the functional variants (or mutants) thereof.

The function of a variant (or mutant) of periostin can be determined according to methods known to those skilled in the art (for review, see Kudo et al., 2011).

The total or partial inhibition of the expression and/or of the activity of periostin can be obtained in various ways, using methods known per se.

This inhibition can be obtained by intervening upstream of the production of periostin, by mutagenesis of the gene encoding this protein, or else by inhibition or modification of the periostin transcription or translation.

The mutagenesis of the gene encoding periostin can take place at the level of the coding sequence or of the sequences for regulating expression, in particular of the promoter. The deletion of all or part of said gene and/or the insertion of an exogenous sequence can, for example, be carried out (see, for example, Rios et al., 2005).

It is also possible to introduce one or more point mutations with physical agents (for example radiation) or chemical agents. The consequence of these mutations is to shift the reading frame and/or to introduce a stop codon into the sequence and/or to modify the level of transcription and/or of translation of the gene and/or to render the periostin less active than wild-type periostin. The mutated alleles of the gene encoding periostin can be identified, for example, by PCR using primers specific for said gene (see, for example, Rios et al., 2005).

A site-directed mutagenesis, targeting a gene encoding said periostin, can also be carried out. The inhibition or the modification of transcription and/or of translation can be obtained by expression of sense, antisense or double-stranded RNAs derived from the gene of said periostin, or of the cDNA of this protein, or else by using interfering RNAs.

The techniques for genetic modifications of cells are known to those skilled in the art. By way of example, Casteilla et al., 2008, describes a method for gene transfer in cells derived from adipose tissue using viral vectors.

According to this embodiment of the present invention, a recombinant DNA construct comprising one or more polynucleotides capable of inhibiting periostin expression can be used. By way of nonlimiting examples, said polynucleotides can encode antisense RNAs, such as morpholino antisense oligonucleotides, hairpin RNAs, interfering RNAs (noncoding double-stranded RNAs approximately 21 to 25 nucleotides in length), shRNAs, micro-RNAs (noncoding single-stranded RNAs approximately 21 to 25 nucleotides in length), aptamers, or ribozymes targeting a gene encoding periostin.

Preferably, said polynucleotide capable of inhibiting periostin expression is an interfering RNA (siRNA).

According to one advantageous arrangement, the siRNA of sequence SEQ ID No. 1 can be used.

Those skilled in the art have available a very wide choice of elements useable for obtaining recombinant DNA constructs in accordance with this embodiment of the invention.

Blocking antibodies directed against periostin, or inhibitors of periostin, can also be used. Such antibodies are described by Zhu et al., 2011 and Orecchia et al., 2011.

The increase in the expression (i.e. overexpression) and/or in the activity of periostin in a cell having an immunomodulatory potential as defined above can be carried out by modifying the genome of said cell, by stimulating the periostin signaling pathways in said cell or by using mediators inducting periostin expression.

This modifying of the genome can in particular be carried out by genetic transformation of said cell with one or more copies of a polynucleotide encoding said periostin, combined with cis regulatory sequences for its expression. The overexpression of said periostin can also be obtained by modifying the cis regulatory sequences for the expression of said periostin, for example by replacing its endogenous promoter with a stronger promoter, allowing a higher level of transcription, or else by attaching, to the endogenous promoter, transcription-activating sequences, of “enhancer” type, or translation-activating sequences.

According to one mode of this embodiment of the present invention, use is made of an expression cassette comprising a polynucleotide encoding a periostin as defined above, placed under the transcriptional control of an appropriate promoter. Said promoter may be a heterologous promoter. In this case, use may be made, for example, of a constitutive promoter, such as the CMV, β-actin, EF1-α, PGK and ubiquitin C promoters, a promoter specific for a given tissue or a locally inducible promoter.

Use may also be made of recombinant vectors, resulting from the insertion of an expression cassette as described above into a host vector.

The expression cassettes and recombinant vectors as described above can, of course, also comprise other sequences, usually employed in constructs of this type. The choice of these other sequences will be made, conventionally, by those skilled in the art according to, in particular, criteria such as the host cells chosen, the transformation protocols envisioned, etc.

By way of nonlimiting examples, mention will be made of transcription terminators and leader sequences. These sequences may be those which are naturally associated with the gene encoding periostin as defined above, or else may be heterologous sequences. These sequences do not affect the specific properties of the promoter or of the gene with which they are associated, but can qualitatively or quantitatively improve, overall, the transcription and, where appropriate, the translation. It is also possible, for the purpose of increasing the expression level, to use transcription and translation enhancer sequences.

Among the other sequences commonly used in the construction of expression cassettes and recombinant vectors, mention will also be made of sequences which make it possible to monitor the transformation, and to identify and/or select the transformed cells.

The stimulation of the periostin signaling pathway can be carried out by stimulating the TGF-β signaling pathway or the bone morphogenetic protein (BMP) signaling pathway in said immunomodulatory cell (for review, see Frangogiannis, 2012).

By way of examples of mediators which induce periostin expression, mention may be made of angiotensin II, and the cytokines IL-4 and IL-13 (for review, see Frangogiannis, 2012).

The culture supernatant of an isolated cell having an immunomodulatory potential, in which the expression and/or the activity of periostin is modulated, as defined above, can be obtained by culturing said cell in an appropriate culture medium, and recovering and filtering the culture supernatant.

A subject of the present invention is also a pharmaceutical composition comprising an isolated cell having an immunomodulatory potential, in which the expression and/or the activity of periostin is modulated, or the culture supernatant of said cell, as defined above, and at least one pharmaceutically acceptable vehicle.

According to one advantageous embodiment of said composition, said pharmaceutically acceptable vehicle is suitable for cell therapy. The preparation of stromal cells for use thereof in cell therapy is well known to those skilled in the art (Le Blanc et al., 2008, Constantin et al., 2009, Garcia-Olmo et al., 2009, Gonzalez et al., 2009a and 2009b and Karussis et al., 2010).

A subject of the present invention is also the use of an isolated cell having an immunomodulatory potential, in which the expression and/or the activity of periostin is modulated, or the culture supernatant of said cell, or of a pharmaceutical composition, as defined above, for the production of an immunosuppressive or immunostimulatory medicament as defined above.

A subject of the present invention is also a method for the regeneration of a tissue or for organ transplantation or for treating or preventing a disease, as defined above, comprising the administration, to said subject, of a therapeutically effective amount of an isolated cell having an immunomodulatory potential, in which the expression and/or the activity of periostin is modulated, or the culture supernatant of said cell, or of a pharmaceutical composition, as defined above.

A subject of the present invention is also the in vitro use of an isolated cell having an immunomodulatory potential, in which the expression and/or the activity of periostin is modulated, as defined above, for identifying (or screening for) a product which modifies the effects of periostin in said cell.

A subject of the present invention is also an in vitro model for carrying out pharmacological or toxicological tests, comprising an isolated cell having an immunomodulatory potential, in which the expression and/or the activity of periostin is modulated, as defined above, for identifying (or screening for) a product which modifies the effects of periostin in said cell.

A subject of the present invention is also
a protein chosen from periostin and a protein comprising the first, second, third and/or fourth FAS1 domain, preferentially the second FAS1 domain, of periostin, preferably periostin,
a nucleic acid molecule comprising a sequence encoding said protein or
a pharmaceutical composition comprising said protein or said nucleic acid molecule, and at least one pharmaceutically acceptable vehicle, for the use thereof as an immunostimulatory medicament intended for vaccination, i.e. a vaccine adjuvant, or in the treatment of a cancer or of an infection associated with an immunodeficiency, of a selective or combined immunoglobulin deficiency, of an isolated T lymphocyte deficiency, of a purine nucleoside phosphorylase deficiency, of a severe combined immunodeficiency caused by adenosine deaminase deficiency, or of common variable hypogammaglobulinemia.

The invention encompasses natural, recombinant or synthetic periostin.

The term “recombinant periostin” is intended to mean periostin produced by genetic engineering, for example by cloning and gene amplification.

The term “synthetic periostin” is intended to mean periostin produced by enzymatic and/or chemical synthesis.

Said periostin may be of human origin as defined above, or of animal origin.

The nucleic acid molecule encoding said protein is obtained by conventional methods, known per se to those skilled in the art, according to standard protocols (see, for example, international application WO 2010/025555).

The said nucleic acid molecule may be in the form of a eukaryotic or prokaryotic recombinant vector, comprising an insert consisting of a polynucleotide encoding periostin. Many vectors into which a polynucleotide of interest can be inserted in order to introduce it into and to maintain it in a eukaryotic or prokaryotic host cell are known per se; the choice of an appropriate vector depends on the use envisioned for this vector (for example, expression of this sequence or integration into the chromosomal material of the host), and also on the nature of the host cell. For example, viral vectors or nonviral vectors, such as plasmids, can be used.

Preferably, said recombinant vector is an expression vector in which said polynucleotide is placed under the control of appropriate regulatory elements for transcription and translation.

The subject of the present invention is also a periostin inhibitor selected from the group consisting of an anti-periostin blocking antibody, an antisense RNA, a morpholino antisense oligonucleotide, a hairpin RNA, an interfering RNA (siRNA), and an aptamer, which are directed against periostin, or a pharmaceutical composition comprising said inhibitor and at least one pharmaceutically acceptable vehicle, for the use thereof as an immunosuppressive medicament for the regeneration of a tissue or for organ transplantation or in the treatment of a disease chosen from the group consisting of graft versus host disease, chronic inflammatory bowel diseases, chronic inflammatory rheumatism, chronic inflammatory diseases of the central nervous system, lupus, autoimmune thyroiditis, complex anal fistulae, asthmatic reactions of type IV delayed hypersensitivity type, inflammatory scars, allergies, tissue necroses, autoimmune diseases, ulcers, diabetes and microbial infections.

The preparation of anti-periostin antibodies is known to those skilled in the art (see application EP 2 168 599 A1). By way of example, of anti-human periostin blocking antibodies, mention may be made of those described by Orecchia et al., 2011 and Zhu et al., 2011.

According to one advantageous arrangement, the siRNA of sequence SEQ ID No. 1 can be used.

By way of nonlimiting examples of a pharmaceutically acceptable vehicle, mention may be made of dispersants, solubilizing agents, stabilizers, preservatives, etc. Pharmaceutically acceptable vehicles useable in (liquid and/or injectable and/or solid) formulations are in particular methylcellulose, hydroxymethylcellulose, carboxymethylcellulose, cyclodextrins, polysorbate 80, mannitol, gelatin, lactose, plant or animal oils, acacia, etc.

Said medicament or said pharmaceutical composition may be in the form of an isotonic and buffered physiological saline solution compatible with pharmaceutical use and known to those skilled in the art.

The amount of said protein or of said periostin inhibitor used as a medicament according to the invention or present in the pharmaceutical composition according to the invention may be modulated so as to obtain a circulating level of active ingredient (in a physiological fluid such as blood) necessary for obtaining the desired therapeutic effect for a particular subject. The amount chosen will depend on many factors, in particular on the route of administration, on the duration of administration, on the moment of the administration, on the rate of elimination of the compound, on the various product(s) used in combination with said medicament or said pharmaceutical composition, on the age, the weight and the physical condition of the patient, and also on the medical history of said patient, and on any other information known in medicine.

The medicament or the pharmaceutical composition according to the present invention may be used alone or in combination with at least one other therapeutically active compound, such as, for example, an antigen or a second immunostimulatory or immunosuppressive compound, according to the desired use of the medicament. The use of said medicament or of said pharmaceutical composition, and of said therapeutically active compound, may be simultaneous, separate or spread out over time.

A subject of the present invention is also a method for the treatment or prevention, in a subject, of a cancer or of an infection associated with an immunodeficiency, of a selective or combined immunoglobulin deficiency, of an isolated T lymphocyte deficiency, of a purine nucleoside phosphorylase deficiency, of a severe combined immunodeficiency caused by adenosine deaminase deficiency or of common variable hypogammaglobulinemia, comprising the administration, to said subject, of a therapeutically effective amount of said pharmaceutical composition comprising said protein (periostin or a protein comprising 1, 2, 3 or 4 FAS1 domains of periostin) or a nucleic acid molecule comprising a sequence encoding said protein, as defined above.

A subject of the present invention is also a method for regeneration of a tissue, for transplantation of an organ or for treatment or prevention of a disease chosen from the group consisting of graft versus host disease, chronic inflammatory bowel diseases, chronic inflammatory rheumatism, chronic inflammatory diseases of the central nervous system, lupus, autoimmune thyroiditis, complex anal fistulae, asthmatic reactions of type IV delayed hypersensitivity type, inflammatory scars, allergies, tissue necroses, autoimmune diseases, ulcers, diabetes, and microbial infections, in a subject, comprising the administration, to said subject, of a therapeutically effective amount of said pharmaceutical composition comprising an anti-periostin blocking antibody, an antisense RNA, a morpholino antisense oligonucleotide, a hairpin RNA, an interfering RNA (siRNA) and/or an aptamer, which are directed against periostin, as defined above.

In addition to the above arrangements, the invention comprises other arrangements, which will emerge from the following description, which refers to examples showing, in vitro, the effect of the modulation of periostin expression in adipose tissue-derived mesenchymal stromal cells (ASCs) on the immunomodulatory potential of these cells, and also to the appended figures, in which:

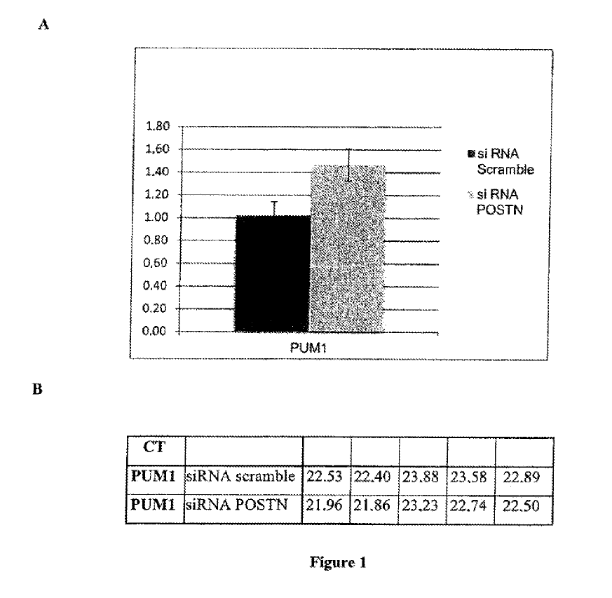

FIG. 1 represents the effect of the siRNA directed against POSTN (siRNA POSTN) and of the control nonspecific siRNA (siRNA scramble) on the amount of mRNA encoding PUM1 (used as a control) in the treated ASCs. A. The mean±standard deviation of the mean (sem) after normalization of the values is represented on the graph. B. The table represents the individual values measured.

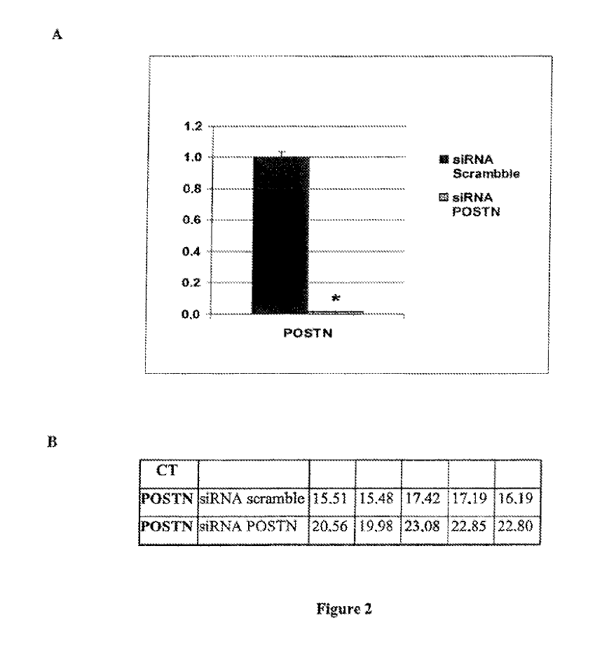

FIG. 2 represents the effect of the siRNA directed against POSTN (siRNA POSTN) and of the control nonspecific siRNA (siRNA scramble) on the amount of mRNA encoding the periostin in the treated ASCs. A. The mean±standard deviation of the mean (sem) is represented after normalization of the values on the graph. B. The table represents the individual values measured.

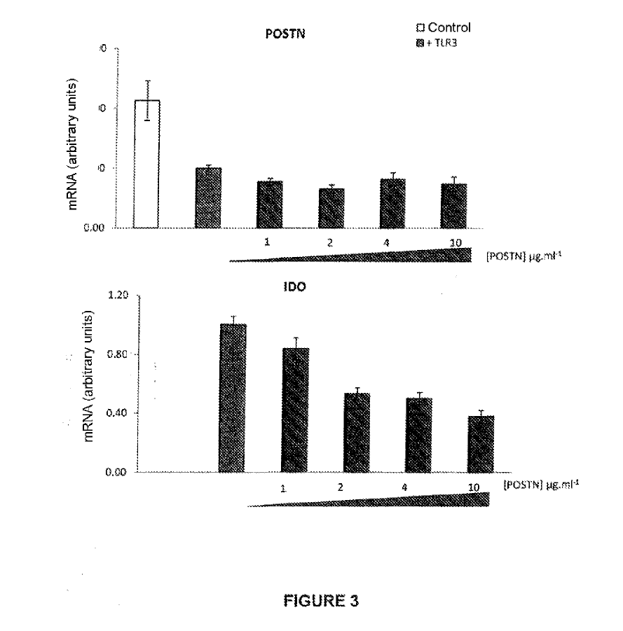

FIG. 3 represents the in vitro effect of the treatment of the ASCs with the TLR3 ligand Poly(I:C) alone or in combination with periostin (POSTN) at a dose of 1, 2, 4 or 10 μg/ml, on the expression of the POSTN mRNA (A) and the IDO1 mRNA (B).

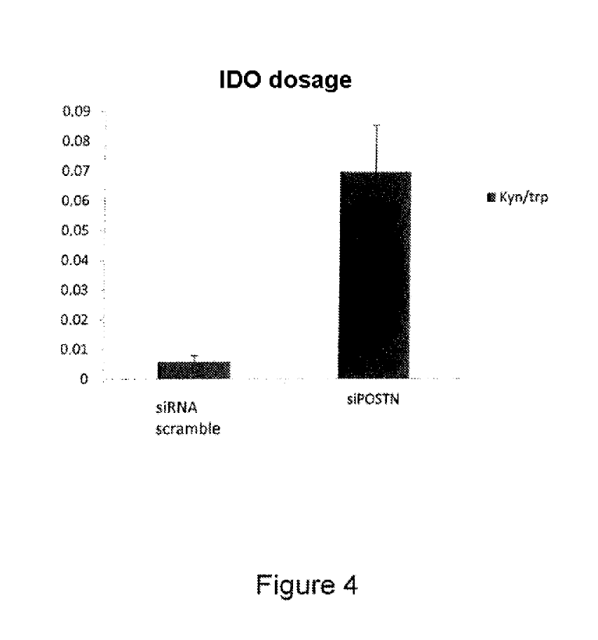

FIG. 4 represents the dosage of IDO-1 in the culture supernatant of ASCs treated with an siRNA directed against POSTN (siRNA POSTN) or a control nonspecific siRNA (siRNA scamble).

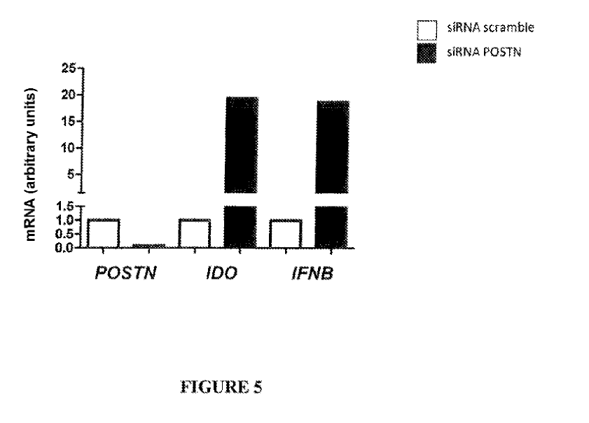

FIG. 5 represents the effect of the siRNA directed against POSTN (siRNA POSTN) and of the control nonspecific siRNA (siRNA scramble) on the amount of mRNA encoding POSTN, IDO1 (IDO) and IFN-β (IFNB) in the treated BM-MSCs.

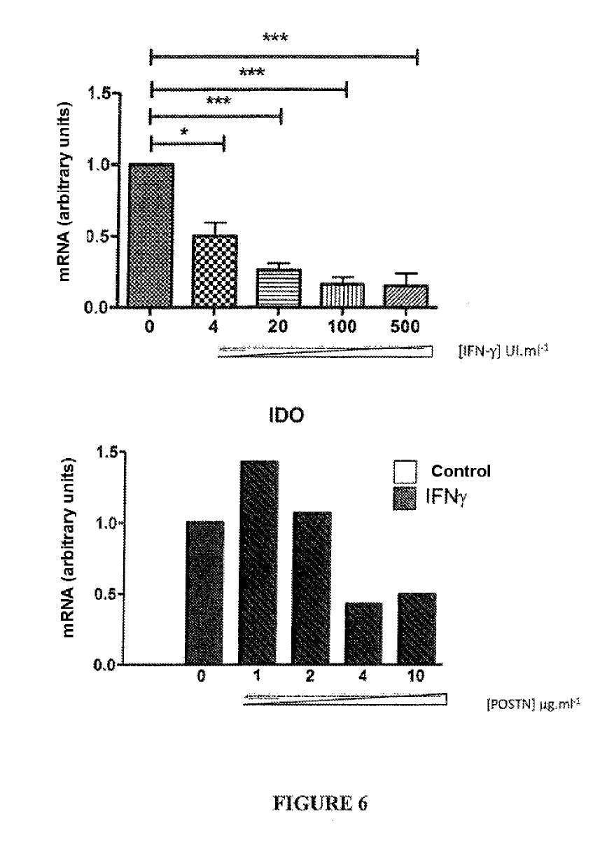

FIG. 6 represents (A) the in vitro effect of the treatment of the ASCs with IFNγ (A) at a dose of 4, 20, 100 or 500 IU/ml on the expression of the POSTN mRNA and (B) the in vitro effect of the treatment of the ASCs with IFNγ at a dose of 100 IU/ml in combination with periostin (POSTN) at a dose of 1, 2, 4 or 10 μg/ml, on the expression of the IDO1 mRNA.

### EXAMPLE 1

### In Vitro Effect of the Inhibition of Periostin Expression in Adipose Tissue-Derived Mesenchymal Stromal Cells (ASCs)

1) Materials and Methods

Isolation of Human Adipose Tissue-Derived Mesenchymal Stromal Cells (ASCs)

The stromal vascular fraction (SVF) was isolated from human subcutaneous adipose tissue by digestion with collagenase NB4 (0.4 U/ml final concentration in α-MEM medium+ciprofloxacin 10 μg/ml final concentration [=α-MEM OK medium]) for 45 min at 37° C. with stirring. The digestion was stopped using cold α-MEM OK medium. The cell suspension was then filtered through a 100 μm nylon membrane. After centrifugation for 10 min at 1600 rpm, the cells were taken up in CPM culture medium (α-MEM OK+1 U/ml heparin+2% of platelet growth factor-enriched plasma) and counted on a Countess® automated device, according to the information from the manufacturer (Life Technologies).

The cells of the SVF were plated out at a density of 4000 cells/cm² in the CPM medium. After 12 h of culture at 37° C. and 5% CO₂, the nonadherent cells were removed by washing with PBS (phosphate buffered saline). The adherent fraction was then placed in culture in vitro in the same CPM culture medium, the medium being renewed three times per week. After 8 days of culture, the ASCs (passage 0) were harvested with trypsin-EDTA (Life Technologies). The number of viable cells was determined by Trypan blue exclusion on a Countess® automated device. The cells were then plated out at a density of 2000 cells/cm² and cultured for a further 2 days (passage 1). The treatment with the siRNA was then carried out.

Isolation of Bone Marrow-Derived Mesenchymal Stromal Cells (BM-MSCs)

Human nuclear bone marrow cells were first seeded at 5×10⁴ cells/cm² in a culture medium consisting of the alpha minimum essential medium (cMEM) supplemented with 10% of filtered fetal calf serum (FCS; Hyclone), 1 ng/ml of fibroblast growth factor 2 (FGF2, R&D Systems, Lille, France) and 10 μg/ml of ciprofloxacin. All the medium was renewed twice a week until the cells reached confluence (end of P0). The cells were then detached using trypsin. The viable cells were counted and reseeded at 500 cells/cm² (passage P1).

Treatment with the siRNA

The siRNA directed against periostin (POSTN) was supplied by Sigma (MISSION esiRNA Human POSTN, EHU069741). This siRNA is directed against the 4 isoforms of human periostin. The nonspecific siRNA AF488 was supplied by Qiagen (siRNA AllStarNeg AF488). After 2 days of culture, the culture medium of the ASCs or of the BM-MSCs was replaced with the culture medium for the treatment with the siRNA, prepared as described below.

Briefly, 2 μl of siRNA at 28 μM were diluted in 100 μl of α-MEM OK medium, vortexed for 10 seconds and then mixed with 12 μl of HiPerfect reagent (Qiagen). After further vortexing, the suspension obtained was left at ambient temperature for 10 min before being mixed with 2 ml of CPM medium. The ASCs were then added to this medium. The cells were then cultured for 4 days in this medium at 37° C. and 5% CO₂.

RNA Extraction

After 4 days of culture, the culture medium of the was removed and the cells were frozen at −80° C. The RNA extraction was carried out according to the manufacturer's instructions (RNeasy minikit, Qiagen). The RNAs were quantified using the Nanodrop automated device (ThermoScientific) and 1 μg of RNA was reverse-transcribed using the SuperScript One Step RT kit according to the recommendations of the manufacturer (LifeTechnologies).

Quantitative RT-PCR (RT-qPCR)

The cDNA quantification was carried out using the StepOnePlus and the Mix SybrGreen technology according to the instructions of the manufacturer (LifeTechnologies).

Primers

The primers used for the RNA quantification are the following:

TABLE 1

| Gene | 5′-3′ | 5′-3′ |
|---|---|---|
| | Sense primer | Antisense primer |
| PUM1 | AGTGGGGGACT | GTTTTCATCAC |
| | AGGCGTTAG | TGTCTGCATCC |
| | (SEQ ID NO. 2) | (SEQ ID NO. 3) |
| IFN-β | CCTGTGGCAAT | GGCGTCCTCCT |
| | TGAATGGG | TCTGGAAC |
| | (SEQ ID NO. 4) | (SEQ ID NO. 5) |
| IDO1 | GCCTGATCTCA | TGCATCCCAGA |
| | TAGAGTCTGGC | ACTAGACGTGC |
| | (SEQ ID NO. 6) | (SEQ ID NO. 7) |
| TSG6 | TCACCTACGCA | TCCAACTCTGC |
| | GAAGCTAAGGC | CCTTAGCCATC |
| | (SEQ ID NO. 8) | (SEQ ID NO. 9) |
| HLA-G | GAAGAGGAGAC | TCGCAGCCAAT |
| | ACGGAACACCA | CATCCACTGGA |
| | (SEQ ID NO. 10) | (SEQ ID NO. 11) |
| IL-IRA | ATGGAGGGAAG | GTCCTGCTTTC |
| | ATGTGCCTGTC | TGTTCTCGCTC |
| | (SEQ ID NO. 12) | (SEQ ID NO. 13) |
| AIRE | CAGACCATGTC | ACCTGGATGCA |
| | AGCTTCAGTCC | CTTCTTGGAGC |
| | (SEQ ID NO. 14) | (SEQ ID NO. 15) |
| POSTN | CAGCAAACCAC | TTAAGGAGGCG |
| | CTTCACGGATC | CTGAACCATGC |
| | (SEQ ID NO. 16) | (SEQ ID NO. 17) |

Pumillo-1 (PUM1) is used as reference gene.

Statistical Analyses

The quantitative RT-PCR results are expressed as mean values±standard deviation of the mean (sem). The analysis of the significance was carried out using the Mann & Whitney test or the Student's t test (Prism 5 software) (*P<0.05, **P<0.01).

Activity of IDO in the Culture Supernatant of ASCs Treated with the siRNA Directed Against POSTN

The activity of IDO-1 (indoleamine 2,3-dioxygenase 1) was determined by high performance liquid chromatography by measuring the kynurenine concentration in the culture supernatant of the ASCs treated with the siRNA directed against POSTN or a nonspecific siRNA, and using 3-nitro-L-tyrosine as internal standard. The kynurenine and the 3-nitro-L-tyrosine were detected by UV absorption at 360 nm.

2) Results

The treatment of the ASCs with the siRNA directed against POSTN does not induce any significant modification of PUM1 expression compared with the treatment with the nonspecific siRNA (see FIG. 1). The nonspecific siRNA can therefore be used as a control siRNA.

The amount of mRNA encoding periostin was then determined by RT-qPCR after treatment of the ASCs with the siRNA directed against POSTN or the nonspecific siRNA. The results are represented in FIG. 2. These results show that the treatment of the ASCs with the siRNA directed against POSTN is effective for inhibiting POSTN expression in these cells.

The amount of mRNA encoding various immunosuppressive proteins was then determined by RT-qPCR after treatment of the ASCs with the siRNA directed against POSTN or the nonspecific siRNA. The results are represented in table 2 below.

TABLE 2
effect of the siRNA directed against POSTN and
of the control nonspecific siRNA on the amount of mRNA
encoding periostin (POSTN) and various
immunosuppressive proteins in the treated ASCs. The
means ± standard deviation after normalization of the
values measured are represented.

| | |
|---|---|
| Nonspecific | |
| siRNA | POSTN siRNA |

| Protein | Mean | sem | Mean | Sem |
|---|---|---|---|---|
| POSTN | 1.00 | 0.00 | 0.08 | 0.03 |
| IFN-β | 1.00 | 0.00 | 289.38 | 120.77 |
| IDO-1 | 1.00 | 0.00 | 92.74 | 72.18 |
| TSG-6 | 1.00 | 0.00 | 1.89 | 0.59 |
| HLA-G | 1.00 | 0.00 | 3.70 | 1.64 |
| IL-IRA | 1.00 | 0.00 | 4.00 | 1.81 |
| AIRE | 1.00 | 0.00 | 2.15 | 0.48 |

These results show that the inhibition of POSTN expression using an interfering RNA strategy induces a very strong increase in the expression of IFN-β (interferon-beta) and of IDO-1 (indoleamine 2,3-dioxygenase 1) and an increase in the expression of TSG-6 (tumor necrosis factor-inducible gene 6 protein), HLA-G (class I, major histocompatibility complex antigen G), IL-1RA (interleukin-1 receptor antagonist) and AIRE (autoimmune regulator).

These results suggest that periostin controls the expression of the genes encoding IFN-β and IDO-1, which have immunosuppressive properties. These results also suggest that periostin controls the immunomodulatory activity of ASCs.

In addition, the inhibition of POSTN expression using an interfering RNA strategy induces a very strong increase in the activity of IDO-1 in the culture supernatant of the ASCs treated with the siRNA directed against POSTN (see FIG. 4).

Insofar as ASCs have characteristics similar to the other mesenchymal stem/stromal cells, and to progenitor cells, precursor cells, cells differentiated from a mesenchymal stromal cell, macrophages, monocytes, mast cells, myeloid cells, fibroblasts, dendritic cells, lymphocytes (for example Treg lymphocytes), NK cells, lymphoid cells and myoblasts, it is probable that periostin also plays a role in the modulation of the immunosuppressive properties of these cells.

Similar results were, moreover, obtained with bone marrow-derived mesenchymal stromal cells (BM-MSCs): the inhibition of POSTN expression using an interfering RNA strategy induced a very strong increase in the expression of IDO-1 and of IFN-β by the BM-MSCs (see FIG. 5). The effect of the modulation of periostin expression on the immunomodulatory potential of cells is therefore not specific to adipose tissue-derived mesenchymal stromal cells (ASCs), but is also exerted in particular on bone marrow-derived mesenchymal stromal cells (BM-MSCs).

Moreover, it has been shown that human mesenchymal stromal cells exhibit indoleamine 2,3-dioxygenase (IDO)-induced antimicrobial effector functions against various pathogens, such as bacteria, protozoan parasites and viruses (Meisel et al., 2011 and Krampera, 2011).

The results obtained above consequently suggest that an isolated cell having an immunomodulatory potential, preferably an ASC, in which the expression and/or the activity of periostin is inhibited, exhibits antimicrobial properties, since IDO-1 expression is significantly increased in said cell.

### EXAMPLE 2

### In Vitro Effect of the Addition of Periostin in ASCs Treated with the TLR3 Ligand

1) Materials and Methods

The isolation of the human adipose tissue-derived mesenchymal stromal cells (ASCs) was carried out as previously described in example 1-1 above, with the exception that the cells of the SVF were plated out at a density of 2000 cells/cm² and cultured for a further 5 days (passage 1) with a change of culture medium after 2 days.

After the 5 days of culture in passage 1, the cells were treated with Poly(I:C), which is a TLR3 ligand (InvivoGen, Poly(I:C)-LMW), and/or periostin (POSTN; R&D Systems, Recombinant Human Periostin/OSF-2). The medium used for the treatment is the CPM medium supplemented with Poly(I:C) at a concentration of 500 μg/ml and/or with periostin (POSTN) at a concentration of 1, 2, 4 or 10 μg/ml.

After 24 hours of treatment, the medium was removed and the cells were frozen at −80° C. The extraction of the IDO1 RNAs and the RT-qPCR were carried out as previously (see example 1-1 above).

2) Results

The results are represented in FIG. 3.

The immunosuppressive properties of the ASCs were stimulated by adding Poly(I:C).

The addition of increasing doses of periostin inhibits the effect of Poly(I:C) on IDO1 expression.

These results show that, when the immunosuppressive effect is stimulated, the addition of POSTN inhibits this effect.

Similar results were obtained when replacing Poly(I:C) with IFNγ (R&D Systems), which is a stimulus capable of also inducing IDO-1 production. Indeed, the addition of IFNγ induced a decrease in POSTN expression in the ASCs (measured by RT-qPCR) in a dose-dependent manner, and the addition of POSTN to the ASC culture supernatant inhibited the IFNγ-induced IDO-1 expression (see FIG. 6). These results show that the effect of POSTN is not specific to TLR3.

Increasing periostin expression in adipose tissue-derived mesenchymal stromal cells (ASCs) therefore makes it possible to decrease the immunosuppressive potential of these cells.

### REFERENCES

Abdelrazik H, et al., Eur. J. Immunol. 2011; 41:3281-90.
Casteilla L, et al., Curr Gene Ther. 2008; 8:79-87.
Casteilla L, et al., World J Stem Cells. 2011; 3:25-33.
Charrière G M, et al., Exp Cell Res. 2006; 312:3205-14.
Constantin G, et al., Stem Cells. 2009; 27:2624-35.
Coutu D L, et al., J Biol Chem. 2008; 283:17991-8001.
Dazzi F et Krampera M, Best Pract Res Clin Haematol. 2011; 24:49-57.
Garcia-Olmo D, et al., Dis Colon Rectum. 2009; 52:79-86.
Gimble J M, et al., Circ Res. 2007; 100:1249-60.
González M A, et al., Gastroenterology. 2009a; 136:978-89.
González M A, et al., Arthritis Rheum. 2009b; 60:1006-19.
Frangogiannis N G, Physiol Rev. 2012; 92:635-88.
Haniffa M A, et al., J Immunol. 2007; 179:1595-604.
Hoogduijn M J, et al., Int Immunopharmacol, 2010; 10; 1496-500.
Karussis D, et al., Arch Neurol. 2010; 67:1187-94.
Kode J A, et al., Cytotherapy. 2009; 11:377-91.
Krampera M, Leukemia. 2011; 25:1408-14.
Kronsteiner B, et al., Stem Cells Dev. 2011; 20:2115-26.
Kudo H, et al., Dev Biol. 2004; 267:473-87.
Kudo A, Cell Mol Life Sci. 2011; 68:3201-7.
Le Blanc K, Cytotherapy. 2006; 8:559-61.
Le Blanc K, et al., Lancet. 2008; 371:1579-86,
Meisel R, et al., Leukemia. 2011; 25:648-54.
Najar M, et al., Cell Immunol. 2010; 264:171-9.
Orecchia P, et al., Eur J Cancer. 2011; 47:2221-9.
Perico N, et al., Clin J Am Soc Nephrol. 2011; 6:412-22.
Planat-Benard V, et al., Circulation. 2004; 109:656-63.
Puissant B, et al., Br J Haematol. 2005; 129:118-29.
Rios H, Mol Cell Biol. 2005; 25:11131-44.
Takayama G, et al., J Allergy Clin Immunol. 2006; 118:98-104.
Uccelli A, et al., Nat Rev Immunol. 2008; 8; 726-736.
Yañez R, et al., Stem Cells. 2006; 24:2582-91.
Zhao Z G, et al., Immunol Invest. 2012; 41:183-98.
Zhou C, et al., Cell Immunol. 2011; 272:33-8.
Zhu M, et al., Mol Cancer Ther. 2011; 10:1500-8.

1. An isolated diploid cell having an immunomodulatory potential, in which the expression, the activity, or the expression and the activity of periostin is modulated, or the culture supernatant of said cell, for the use thereof as a medicament.

2. The cell or supernatant for the use thereof as claimed in claim 1, wherein the expression, the activity, or the expression and the activity of said periostin is totally or partially inhibited.

3. The cell or supernatant for the use thereof as claimed in claim 2, wherein said medicament is an immunosuppressive medicament for the regeneration of a tissue or for organ transplantation or in the treatment of a disease chosen from the group consisting of graft versus host disease, chronic inflammatory bowel diseases, chronic inflammatory rheumatism, chronic inflammatory diseases of the central nervous system, lupus, autoimmune thyroiditis, complex anal fistulae, asthmatic reactions of type IV delayed hypersensitivity type, inflammatory scars, allergies, tissue necroses, autoimmune diseases, ulcers, diabetes and microbial infections.

4. The cell or supernatant for the use thereof as claimed in claim 1, wherein the expression, the activity, or the expression and the activity of said periostin is totally or partially inhibited using a compound chosen from the group consisting of a blocking antibody, an antisense RNA, a morpholino antisense oligonucleotide, a hairpin RNA, an interfering RNA and an aptamer, which are directed against periostin, and a periostin inhibitor.

5. The cell or supernatant for the use thereof as claimed in claim 5, wherein said compound is an siRNA directed against periostin.

6. The cell or supernatant for the use thereof as claimed in claim 1, wherein the expression, the activity, or the expression and activity of said periostin is increased.

7. The cell or supernatant for the use thereof as claimed in claim 6, wherein the increase in the expression, the activity, or the expression and activity of said periostin is carried out by modifying the genome of said cell, by stimulating the periostin signaling pathway in said cell or by using mediators which induce periostin expression.

8. The cell or supernatant for the use thereof as claimed in claim 6, wherein said medicament is an immunostimulatory medicament intended for vaccination or in the treatment of a disease chosen from the group consisting of a cancer or an infection associated with an immunodeficiency, a selective or combined immunoglobulin deficiency, an isolated T lymphocyte deficiency, a purine nucleoside phosphorylase deficiency, a severe combined immunodeficiency caused by adenosine deaminase deficiency, and common variable hypogammaglobulinemia.

9. The cell or supernatant for the use thereof as claimed in claim 1, wherein said cell in which periostin expression is modulated is a human cell.

10. The cell or supernatant for the use thereof as claimed in claim 1, wherein said cell in which periostin expression is modulated is chosen from the group consisting of a mesenchymal stromal cell, a progenitor cell, a precursor cell, a cell differentiated from a mesenchymal stromal cell, a macrophage, a monocyte, a mast cell, a myeloid cell, a fibroblast, a dendritic cell, a lymphocyte, an NK cell, a lymphoid cell and a myoblast.

11. The cell or supernatant for the use thereof as claimed in claim 10, wherein said mesenchymal stromal cell is a mesenchymal stromal cell derived from bone marrow, from adipose tissue, from a solid tissue, from the placenta, from adult blood or from cord blood.

12. The cell or supernatant for the use thereof as claimed in claim 1, wherein said cell having an immunomodulatory potential expresses IFN-β, IDO-1, TSG-6, HLA-G, PGE2, TGF-β, galectin, HO-1, IL-6, IL-1RA, IL-33, AIRE, hEGF, TNF, GM-CSF JAG1, or a combination thereof.

13. A pharmaceutical composition comprising a diploid cell having an immunomodulatory potential, in which the expression, the activity, or the expression and activity of periostin is modulated, or the culture supernatant of said cell, as defined in claim 1, and at least one pharmaceutically acceptable vehicle.

14. An in vitro model for carrying out pharmacological or toxicological tests, comprising a diploid cell having an immunomodulatory potential, in which the expression, the activity, or the expression and activity of periostin is modulated, as defined in claim 1, for identifying a product which modifies the effects of periostin in said cell.

15. The in vitro use of an isolated diploid cell having an immunomodulatory potential, in which the expression, the activity, or the expression and activity of periostin is modulated, as defined in claim 1, for identifying a product which modifies the effects of periostin in said cell.

16. A protein chosen from periostin and a protein comprising at least one of the first, second, third and fourth FAS1 domain of periostin, or a nucleic acid molecule comprising a sequence encoding said protein, or a pharmaceutical composition comprising said protein or said nucleic acid molecule and at least one pharmaceutically acceptable vehicle, for the use thereof as an immunostimulatory medicament intended for vaccination or in the treatment of a cancer or of an infection associated with an immunodeficiency, of a selective or combined immunoglobulin deficiency, of an isolated T lymphocyte deficiency, of a purine nucleoside phosphorylase deficiency, of a severe combined immunodeficiency caused by adenosine deaminase deficiency, or of common variable hypogammaglobulinemia.

17. A periostin inhibitor selected from the group consisting of an anti-periostin blocking antibody, an antisense RNA, a morpholino antisense oligonucleotide, a hairpin RNA, an interfering RNA and an aptamer, which are directed against periostin, or a pharmaceutical composition comprising said inhibitor and at least one pharmaceutically acceptable vehicle, for the use thereof as an immunosuppressive medicament for the regeneration of a tissue or for organ transplantation or in the treatment of a disease chosen from the group consisting of graft versus host disease, chronic inflammatory bowel diseases, chronic inflammatory diseases of the central nervous system, lupus, autoimmune thyroiditis, complex anal fistulae, asthmatic reactions of type IV delayed hypersensitivity type, allergies, autoimmune diseases, ulcers, diabetes and microbial infections.

18. A method for suppressing the immune system of a subject in need thereof, wherein said method comprises administering to said subject an isolated diploid cell having an immunomodulatory potential, in which the expression, the activity, or the expression and the activity of periostin is totally or partially inhibited, or the culture supernatant of said cell.

19. The method of claim 18, wherein said subject is in need of regeneration of a tissue or organ transplantation or the treatment of a disease chosen from the group consisting of graft versus host disease, chronic inflammatory bowel diseases, chronic inflammatory rheumatism, chronic inflammatory diseases of the central nervous system, lupus, autoimmune thyroiditis, complex anal fistulae, asthmatic reactions of type IV delayed hypersensitivity type, inflammatory scars, allergies, tissue necroses, autoimmune diseases, ulcers, diabetes and microbial infections.

20. A method for suppressing the immune system of a subject in need thereof, wherein said method comprises administering to said subject a periostin inhibitor selected from the group consisting of an anti-periostin blocking antibody, an antisense RNA, a morpholino antisense oligonucleotide, a hairpin RNA, an interfering RNA and an aptamer, which are directed against periostin.

21. The method of claim 20, wherein said subject is in need of regeneration of a tissue or organ transplantation or the treatment of a disease chosen from the group consisting of graft versus host disease, chronic inflammatory bowel diseases, chronic inflammatory rheumatism, chronic inflammatory diseases of the central nervous system, lupus, autoimmune thyroiditis, complex anal fistulae, asthmatic reactions of type IV delayed hypersensitivity type, inflammatory scars, allergies, tissue necroses, autoimmune diseases, ulcers, diabetes and microbial infections.

22. A method for stimulating the immune system of a subject in need thereof, said method comprising administering to said subject an isolated diploid cell having an immunomodulatory potential, in which the expression, the activity, or the expression and the activity of periostin is increased, or the culture supernatant of said cell

23. The method of claim 22, wherein said subject is in need of vaccination or the treatment of a disease chosen from the group consisting of a cancer or an infection associated with an immunodeficiency, a selective or combined immunoglobulin deficiency, an isolated T lymphocyte deficiency, a purine nucleoside phosphorylase deficiency, a severe combined immunodeficiency caused by adenosine deaminase deficiency, and common variable hypogammaglobulinemia.

24. A method for stimulating the immune system of a subject in need thereof, said method comprising administering to said subject a protein chosen from periostin and a protein comprising at least one of the first, second, third and fourth FAS1 domain of periostin, or a nucleic acid molecule comprising a sequence encoding said protein, or a pharmaceutical composition comprising said protein or said nucleic acid molecule and at least one pharmaceutically acceptable vehicle.

25. The method of claim 24, wherein the subject is in need of vaccination or the treatment of a cancer or of an infection associated with an immunodeficiency, of a selective or combined immunoglobulin deficiency, of an isolated T lymphocyte deficiency, of a purine nucleoside phosphorylase deficiency, of a severe combined immunodeficiency caused by adenosine deaminase deficiency, or of common variable hypogammaglobulinemia.


SEQUENCE LISTING (17 sequences)

SEQ ID NO: 1 - DNA, 75 bp, Artificial Sequence
  ARNi
aatcatccatgggaaccagattgcaacaaatggtgttgtccatgtcattgaccgtgtgcttacacaaattggtac

SEQ ID NO: 2 - DNA, 20 bp, Artificial Sequence
  Primer
agtgggggactaggcgttag

SEQ ID NO: 3 - DNA, 22 bp, Artificial Sequence
  Primer
gttttcatcactgtctgcatcc

SEQ ID NO: 4 - DNA, 19 bp, Artificial Sequence
  Primer
cctgtggcaattgaatggg

SEQ ID NO: 5 - DNA, 19 bp, Artificial Sequence
  Primer
ggcgtcctccttctggaac

SEQ ID NO: 6 - DNA, 22 bp, Artificial Sequence
  Primer
gcctgatctcatagagtctggc

SEQ ID NO: 7 - DNA, 22 bp, Artificial Sequence
  Primer
tgcatcccagaactagacgtgc

SEQ ID NO: 8 - DNA, 22 bp, Artificial Sequence
  Primer
tcacctacgcagaagctaaggc

SEQ ID NO: 9 - DNA, 22 bp, Artificial Sequence
  Primer
tccaactctgcccttagccatc

SEQ ID NO: 10 - DNA, 22 bp, Artificial Sequence
  Primer
gaagaggagacacggaacacca

SEQ ID NO: 11 - DNA, 22 bp, Artificial Sequence
  Primer
tcgcagccaatcatccactgga

SEQ ID NO: 12 - DNA, 22 bp, Artificial Sequence
  Primer
atggagggaagatgtgcctgtc

SEQ ID NO: 13 - DNA, 22 bp, Artificial Sequence
  Primer
gtcctgctttctgttctcgctc

SEQ ID NO: 14 - DNA, 22 bp, Artificial Sequence
  Primer
cagaccatgtcagcttcagtcc

SEQ ID NO: 15 - DNA, 22 bp, Artificial Sequence
  Primer
acctggatgcacttcttggagc

SEQ ID NO: 16 - DNA, 22 bp, Artificial Sequence
  Primer
cagcaaaccaccttcacggatc

SEQ ID NO: 17 - DNA, 22 bp, Artificial Sequence
  Primer
ttaaggaggcgctgaaccatgc